(19) United States
(12) Patent Application Publication
(10) Pub. No.: US 2026/0013704 A1
(43) Pub. Date: Jan. 15, 2026

(54) Endoscope System with a Rotatably-Adjustable Coupler Cover for a Ring Configured to Adjust an Imaging Parameter

(71) Applicant: ENTELLUS MEDICAL, INC., Maple Grove, MN (US)

(72) Inventor: Paul R. Lesch, JR., Lino Lakes, MN (US)

(21) Appl. No.: 19/115,042

(22) PCT Filed: Sep. 20, 2023
(86) PCT No.: PCT/US2023/033196

§ 371 Date: Mar. 25, 2025

Related U.S. Application Data
(60) Provisional application No. 63/410,038, filed on Sep. 26, 2022.

(51) Int. Cl. A61B 1/00; A61B 1/06

(52) U.S. Cl. CPC A61B 1/00128; A61B 1/00137; A61B 1/00188; A61B 1/0676

(57) ABSTRACT

An example endoscopic camera assembly includes: a camera housing having a proximal end and a distal end; a ring coupled to the distal end of the camera housing; and a coupler cover extending around the ring, wherein the coupler cover is configured to prevent access to a first portion of the ring, wherein the coupler cover comprises one or more windows that provide access to a second portion of the ring, and wherein the ring is rotatable relative to the camera housing and the coupler cover, such that rotation of the ring adjusts an imaging parameter of the endoscopic camera assembly.

### CROSS-REFERENCE TO RELATED APPLICATION

This application claims priority to U.S. Patent Application No. 63/410,038 filed Sep. 26, 2022, which is incorporated herein by reference in its entirety.

### BACKGROUND

The present disclosure relates generally to endoscope systems and components thereof. Endoscopes can be used to visualize a scene of interest during a procedure. Particularly, a light source may be used to illuminate an internal cavity of a patient, and a camera receives reflected light and displays the scene of interest within the internal cavity to a healthcare professional conducting a procedure.

An endoscope system may have input devices to adjust imaging parameters (e.g., focal depth, resolution, aperture, exposure time, etc.) of the camera. In existing endoscope systems, such input devices can be unintentionally triggered by users due to their positioning close to where the user grips the endoscope. Thus, there remains a need for new and improved endoscope systems, devices, and techniques that provide a more ergonomic user experience during endoscopy while precluding unintentional adjustment of the imaging parameters. It is with respect to these and other considerations that the disclosure made herein is presented.

### SUMMARY

Within examples described herein, systems and methods for an endoscope system with a rotatably-adjustable coupler cover for a ring configured to adjust an imaging parameter.

Within additional examples described herein, systems and methods are described that relate to an endoscopic camera assembly having (i) an input device configured to adjust an imaging parameter of the camera, and (ii) a coupler cover extending around the input device to prevent access to a first portion of the input device, while allowing access to a second portion of the input device.

The features, functions, and advantages that have been discussed can be achieved independently in various examples or may be combined in yet other examples. Further details of the examples can be seen with reference to the following description and drawings.

### BRIEF DESCRIPTION OF THE FIGURES

The novel features believed characteristic of the illustrative examples are set forth in the appended claims. The illustrative examples, however, as well as a preferred mode of use, further objectives and descriptions thereof, will best be understood by reference to the following detailed description of an illustrative example of the present disclosure when read in conjunction with the accompanying Figures.

FIG. 1A illustrates a perspective view of an endoscopic camera system, in accordance with an example implementation.

FIG. 1B illustrates a partial perspective view of an endoscopic camera assembly, in accordance with an example implementation.

FIG. 2 illustrates a partial perspective view of the endoscopic camera system of FIG. 1, in accordance with an example implementation.

FIG. 3 illustrates another partial perspective view of the endoscopic camera system of FIG. 1, in accordance with an example implementation.

FIG. 4A illustrates a perspective view of a coupler cover, in accordance with an example implementation.

FIG. 4B illustrates a perspective view of the coupler cover of FIG. 4A from another angle, in accordance with an example implementation.

FIG. 4C illustrates a side view of the coupler cover of FIGS. 4A-4B, in accordance with an example implementation,

FIG. 4D illustrates a side view of the coupler cover of FIGS. 4A-4C with a door covering a window formed in the coupler cover, in accordance with an example implementation.

FIG. 5 illustrates a partial perspective exploded view of an endoscopic camera assembly, in accordance with an example implementation.

FIG. 6 is a flowchart of a method for operating an endoscopic camera system, in accordance with an example implementation.

FIG. 7 is a flowchart of additional operations that are executable with the method of FIG. 6, in accordance with an example implementation.

FIG. 8 is a flowchart of additional operations that are executable with the method of FIG. 6, in accordance with an example implementation.

FIG. 9 is a flowchart of additional operations that are executable with the method of FIG. 6, in accordance with an example implementation.

FIG. 10 is a flowchart of additional operations that are executable with the method of FIG. 6, in accordance with an example implementation.

FIG. 11 is a flowchart of additional operations that are executable with the method of FIG. 6, in accordance with an example implementation.

FIG. 12 is a flowchart of additional operations that are executable with the method of FIG. 6, in accordance with an example implementation.

FIG. 13 is a flowchart of additional operations that are executable with the method of FIG. 6, in accordance with an example implementation.

### DETAILED DESCRIPTION

Endoscopy can be used during a medical procedure to visualize a scene of interest and aid a healthcare professional in conducting the procedure. For example, endoscopy can be used to facilitate positioning of a balloon dilation catheter during a procedure to treat sinusitis. Endoscope systems having improved ergonomics can facilitate proper visualization and can contribute to improved patient outcomes.

An example endoscope camera system can have input devices that enable a user to adjust imaging parameters of a camera. For example, an endoscope camera system may have a focus ring that can be used to adjust a focal depth of the camera as desired by the user. In many cases, such focus ring is positioned close to a grip region of the endoscope (i.e., a region of the endoscope at which the user grips the endoscope during the procedure). As such, the focus ring is prone to unintentional movement as the user grips the endoscope, thereby causing the focal depth of the camera to change inadvertently.

Implementations described herein are relevant to improving user control and ergonomics of an endoscope camera system by providing an improved grip region, where the user can grip the endoscope camera system without accidental triggering of an input device configured to adjust an imaging parameter. Particularly, in an example, disclosed herein is an endoscopic camera assembly with a camera housing having a proximal end and a distal end, and a ring coupled to the distal end of the camera housing, wherein the ring is configured to adjust an imaging parameter of a camera. The endoscopic camera assembly further includes a coupler cover extending around the ring, wherein the coupler cover is configured to prevent access to a first portion of the ring to prevent accidental movement of the ring, while providing access to the ring to allow for adjustment of an angular or rotary position of the ring when desired. The terms “angular position” and “rotary position” are used interchangeably throughout herein.

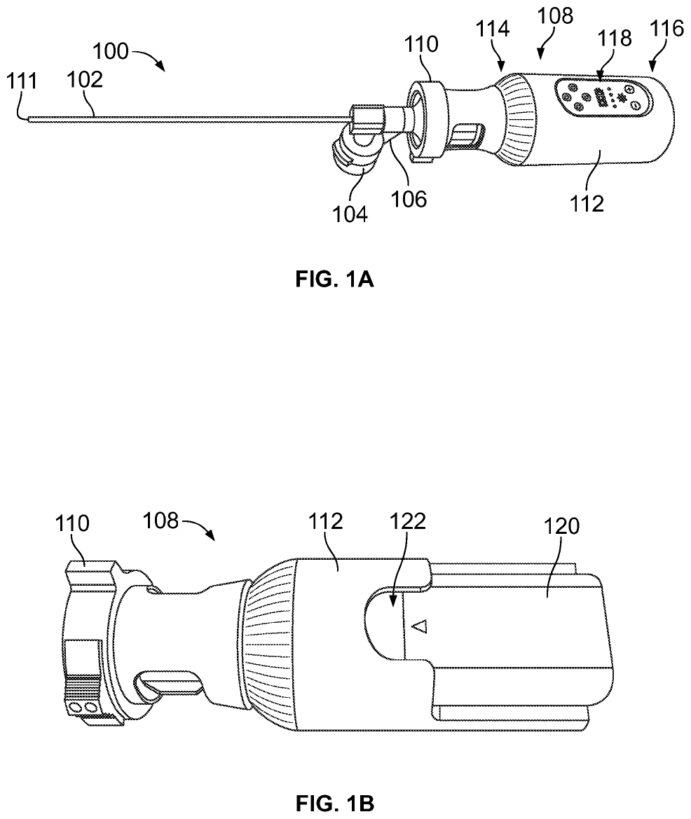

FIG. 1A illustrates a perspective view of an endoscopic camera system 100, in accordance with an example implementation. The endoscopic camera system 100 includes an endoscope 102, a light source 104, a cable 106, and an endoscopic camera assembly 108. The endoscopic camera system 100 further includes a coupling 110 that couples the endoscope 102 to the endoscopic camera assembly 108. In some examples, however, the endoscopic camera system 100 might not include the cable 106. Rather, power can be provided to the light source 104 via the coupling 110.

The endoscope 102 guides light from a distal end of endoscope 102 near a scene of interest to a proximal end of the endoscope 102 to provide a view for capture by a camera within the endoscopic camera assembly 108. Particularly, the endoscope 102 includes a port for receiving illumination from light source 104, which the endoscope 102 uses to illuminate the scene of interest adjacent a distal end 111 of the endoscope 102. In other examples, however, the light source 104 may be included within the coupling 110 and/or the endoscope 102. In examples, the endoscope 102 may be flexible or rigid and may have any of a variety shapes and sizes.

In an example, the light source 104 includes light emitting diodes (LEDs) or other sources of illumination. Further, the light source 104 can include associated driver circuitry, which is located in the endoscopic camera assembly 108 or is external thereto. In an example, the light source 104 can include its own power source or can receive power from an external source, such as from the endoscopic camera assembly 108 via the cable 106.

The endoscopic camera assembly 108 captures a view (e.g., a view transmitted by the endoscope 102) as video, images, or other data. The endoscopic camera assembly 108 includes a camera housing 112 having a distal end 114 and a proximal end 116, and includes a camera disposed within the camera housing 112 and configured to capture the view transmitted by the endoscope 102. Particularly, the camera is a portion of the endoscopic camera assembly 108 that converts visual information received from the endoscope 102 into electrical information.

The endoscopic camera assembly 108 also includes a control panel 118, which can include one or more buttons, switches, slides, dials, or other input mechanisms for controlling the operation of endoscopic camera system 100. Controls can include a power control, an illumination control, a white balance control, a zoom control, controls for wireless transmission settings, and/or other controls.

The endoscopic camera assembly 108 further includes a memory and one or more processors (e.g., computer processing units (CPUs) or other circuitry) for performing processing tasks. The memory may be implemented using any suitable electronically accessible memory, including but not limited to RAM, ROM, Flash, SSD, or hard drives. The memory includes executable instructions that, when executed by the one or more processors, perform tasks associated with operating the endoscopic camera system 100 (e.g., controlling and adjusting illumination, capturing and processing images, transmitting images, etc.).

FIG. 1B illustrates a partial perspective view of the endoscopic camera assembly 108, in accordance to an example implementation. The camera housing 112 can also include a battery 120 configured as a power source for the endoscopic camera assembly 108, and the battery 120 can also power the light source 104. In an example, the camera housing 112 can include a battery cover that is removed in FIG. 1B to provide access to the battery 120.

The camera housing 112 can further include a thumb groove or divot 122. To remove the battery 120, a user can lift or slide the battery 120 outward via a thumb finger (or any other finger) placed in the divot 122. The battery 120 can then be recharged, and returned to the camera housing 112. The camera housing 112 can further include a wireless transmitter that facilitates communication between the endoscopic camera system 100 and other systems (e.g., a remote display).

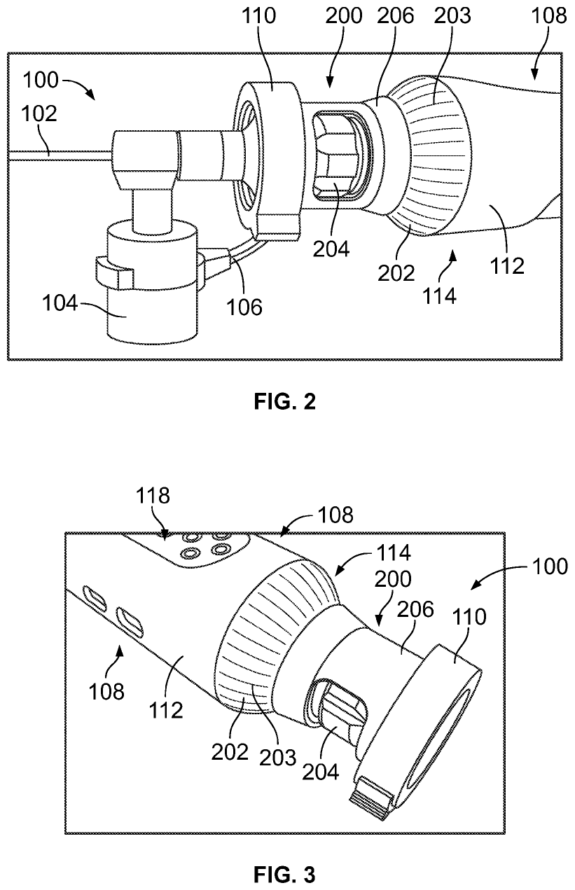

FIG. 2 illustrates a partial perspective view of the endoscopic camera system 100, and FIG. 3 illustrates another partial perspective view of the endoscopic camera system 100, in accordance with an example implementation. The endoscopic camera system 100 includes a grip region 200 configured to be held by a user. For example, the grip region 200 is configured to provide an ergonomic location for use of the endoscopic camera system 100. In an example, the grip region 200 is located at or near a center of gravity of a balance point of the endoscopic camera system 100 to facilitate manipulation of endoscopic camera system 100 by a user.

As illustrated, the camera housing 112 has a conical portion 202 at the distal end 114 of the camera housing 112. In an example, the conical portion 202 can be a separate collar that is mounted to the remaining, cylindrical portion of the camera housing 112. In another example, the conical portion 202 is an integral part of the camera housing 112 (e.g., the conical portion 202 and the cylindrical portion of the camera housing 112 can be formed as a single, monolithic structure).

As depicted, the conical portion 202 can have a plurality of longitudinal grooves 203 formed in a circular or circumferential array about an exterior surface of the conical portion 202. The plurality of grooves 203 may enhance the ease with which the endoscopic camera system 100 is physically handled, lifted, or gripped by a user and may prevent slippage of the endoscopic camera system 100 from the hand of a user. Other techniques could be used. For instance, the conical portion 202 could be knurled. The grip region 200 is defined between the camera housing 112 and the coupling 110, and can include the conical portion 202 of the camera housing 112.

The endoscopic camera system 100 further includes an input device such as a ring 204 disposed in the grip region 200 and coupled to the distal end 114 of the camera housing 112. For example, the ring 204 is configured as a wheel with ridges that facilitate rotating the ring 204, and the ring 204 is used to adjust an imaging parameter of the camera of the endoscopic camera assembly 108. For example, rotating the ring 204 adjusts one of the focal depth, resolution, aperture, exposure time, etc. of the camera. If the ring 204 is used to adjust focal depth of the camera, the ring 204 could be referred to as a focus ring, for example.

As the ring 204 is disposed in the grip region 200, the ring 204 can be unintentionally actuated (i.e., rotated) while handling the endoscopic camera system 100. In other words, the user can adjust the imaging parameter by rotating the ring 204 to a particular rotary position, but can then inadvertently move the ring 204 again, off its desired position, while handling the endoscopic camera system 100.

To mitigate such inadvertent movement of the ring 204, the endoscopic camera assembly 108 includes a coupler cover 206 disposed in the grip region 200, interposed between the conical portion 202 of the camera housing 112 and the coupling 110, and extending around the ring 204. The coupler cover 206 is configured to provide access to the ring 204 to allow for adjusting the imaging parameter via the ring 204 when desired, while at the same time preventing inadvertent access to the ring 204 during handling of the endoscopic camera system 100.

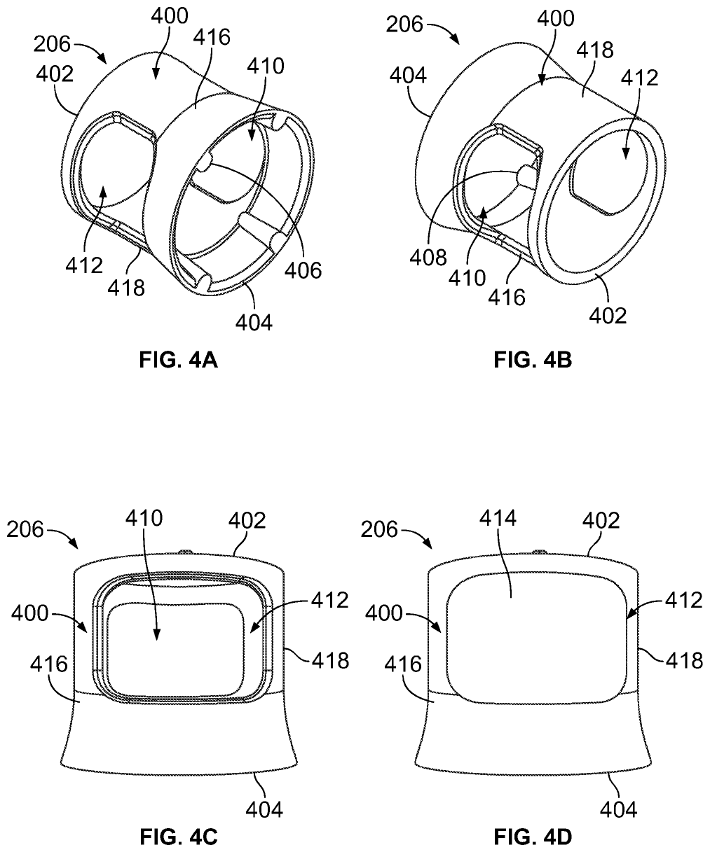

FIG. 4A illustrates a perspective view of the coupler cover 206, FIG. 4B illustrates a perspective view of the coupler cover 206 from another angle, and FIG. 4C illustrates a side view of the coupler cover 206, in accordance with an example implementation. FIGS. 4A-4C are described together.

The coupler cover 206 has a body 400 that is generally shaped as a hollow cylinder. The body 400 has a distal end face 402 configured to interface with the coupling 110 and a proximal end face 404 configured to interface with the conical portion 202 of the camera housing 112.

In an example, the body 400 of the coupler cover 206 is funnel-shaped. Particularly, a proximal portion of the body 400 is divergent such that a diameter of the proximal end face 404 is larger than a respective diameter of the distal end face 402. This configuration allows the coupler cover 206 to conform to a conical surface of the conical portion 202 of the camera housing 112.

In one example, the proximal portion of the coupler cover 206 includes one or more ridges, such as ridge 406 and ridge 408, which extend along a portion of an interior surface of the body 400 in a circular array about the interior surface of the body 400. Such ridges operate as teeth protruding radially-inward from the interior surface of the body 400, and may facilitate gripping the conical portion 202 of the camera housing 112, such that the coupler cover 206 is stably held to the camera housing 112. However, applying a rotational force on the coupler cover 206 allows the coupler cover 206 to overcome friction between the coupler cover 206 and the camera housing 112 to allow the coupler cover 206 to rotate to another desired rotary position.

As depicted, the coupler cover 206 can have one or more holes, cut-outs, or windows, such as first window 410 and second window 412, formed on an exterior peripheral (side) surface of the body 400. In an example, the windows 410, 412 have a pill shape (e.g., each window may be constructed of a rectangle with semicircles at a pair of opposite sides).

In an example, each of the windows 410, 412 has an axial length in a dimension extending between the proximal end face 404 and the distal end face 402 of the coupler cover 206. Each of the windows 410, 412 also has arc length in a dimension extending around a circumference of the coupler cover 206. In examples, the arc length is greater than the axial length.

The windows 410, 412 allow the user to have access to exposed portions of the ring 204. Particularly, the user can use one or more fingers through the windows 410, 412 in the coupler cover 206 to access the ring 204 and rotate it.

At the same time, the coupler cover 206 reduces the likelihood that the ring 204 is unintentionally rotated by accidental contact of the fingers with the ring 204 during handling the endoscopic camera system 100. For example, the coupler cover 206 can be rotated as mentioned above such that the windows 410, 412 are oriented in a manner that reduces the chance that fingers of the user would interact with the ring 204 unintentionally. The rest of the body 400 also protects unexposed parts of the ring 204 from being inadvertently touched by the user.

In the example implementation of FIGS. 4A-4C, the coupler cover 206 includes a pair of oppositely disposed windows (e.g., the windows 410, 412) to enable adjusting or rotating the ring 204 by oppositely located finger tips. In other words, a center of the first window 410 and a center of the second window 412 are diametrically-opposite each other on a circumference of the coupler cover 206. In this example, having diametrically-opposite windows to allow two opposed fingers to simultaneously engage the ring 204 may reduce the force applied by any single finger to rotate the ring 204.

In an example, each of the windows 410, 412 can span a particular angular range about the circumference or the exterior surface of the coupler cover 206. As an example for illustration, each window can have an angular span between 10 degrees and 80 degrees about the exterior peripheral surface of the body 400.

In one example, the windows 410, 412 can be covered to preclude access to the ring 204 until a user desires to access the ring 204 to adjust the imaging parameter of the camera. For example, the coupler cover 206 can include one or more doors that are movable relative to the windows 410, 412 of the coupler cover 206, such that each door is moveable relative to a respective window of the windows 410, 412 between (i) an open position in which the door does not cover the respective window and (ii) a closed position in which the door covers the respective window.

FIG. 4D illustrates a side view of the coupler cover 206 with a door 414 covering the window 412, in accordance with an example implementation. As shown, the door 414 can preclude access to the ring 204 when closed. If a user desires to adjust the ring 204, the user opens the door 414, accesses the ring 204, adjusts the rotary position of the ring 204 as desired, then closes the door 414 again. Each window might have a corresponding door.

Referring to any of FIGS. 4A-4D, the coupler cover 206 has a curved surface 416 and a curved surface 418 interposed between the windows 410, 412. In other words, the curved surfaces 416, 418 interleave with the windows 410, 412 about the exterior peripheral surface of the body 400.

The curves surfaces 416, 418 facilitate resting of fingers of the user in the grip region 200 where the ring 204 is located without engaging the ring 204. Particularly, the curved surfaces 416, 418 allow the fingers to rest comfortably, with less risk of the fingers sliding to either side. In on example, the coupler cover 206 is configured such that the surfaces between the windows 410, 412 include one or more flat or substantially planar grip portions to facilitate resting the fingers of the user in the grip region 200 and gripping the endoscopic camera system 100.

As mentioned above, the coupler cover 206 has ridges (e.g., the ridges 406, 408) that operate as teeth configured to resist rotation of the coupler cover 206 from a particular rotary position relative to the camera housing 112 during operation of the endoscopic camera system 100. The geometry of the coupler cover 206 and the ridges/teeth, however, allow the coupler cover 206 and its windows to be rotatably repositioned as desired by the user without undue effort.

Other configurations can be used to couple the coupler cover 206 to the camera housing 112, while allowing the rotational position of the coupler over 206 to be changed when desired. For example, a coupler cover may include splines formed in an interior or exterior surface thereof, and the camera housing (e.g., the conical portion 202) may include corresponding splines. Such splines can allow the coupler cover to slide into the camera housing 112 and maintain its rotation position. When a user wants to change the orientation of the windows 410, 412, the coupler cover can be pulled, rotated, then slid back into the camera housing 112.

In another example, detents can be used. For example, the camera housing 112 (e.g., the conical portion 202) or the coupler cover 206 can have a plurality of detents that define a plurality of discrete angular positions of the coupler cover 206 relative to the camera housing 112. For instance, the camera housing may have a bored cylinder with a ball configured to slide in the cylinder against a spring. The coupler cover may have a hole formed in its proximal end face having a smaller diameter than the ball. When the hole is in line with the cylinder, the ball is pushed by the spring to fall partially into the hole under spring force, holding the coupler cover to the camera housing. The coupler cover may have several holes in its proximal end face to allow for changing the rotary position of the coupler cover and the windows as desired by a user.

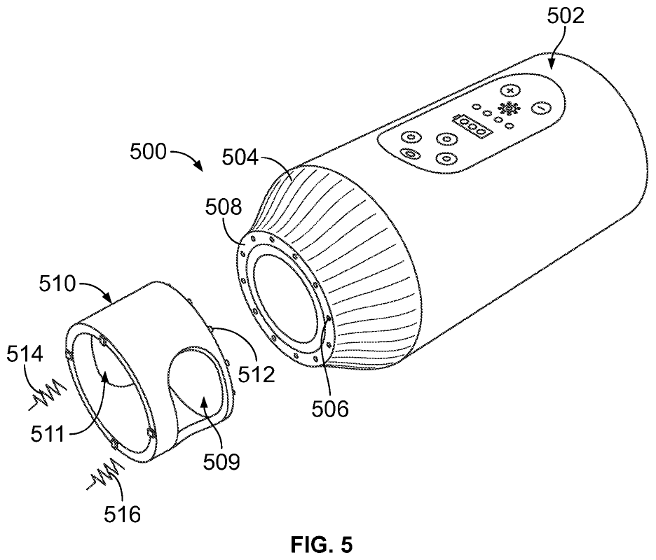

In another example, one of the coupler cover and the camera housing includes a plurality of pins or posts, and the other one of the coupler cover and the camera housing comprises a plurality of detents or receptacles, wherein the plurality of receptacles are configured to receive the plurality of posts. In this example, the coupler cover is configured to move between a proximal position relative to the camera housing and a distal position relative to the camera housing. In the proximal position, the plurality of posts are received in the plurality of receptacles to prevent rotation of the coupler cover relative to the camera housing during operation of the endoscopic camera system 100. In the distal position, the plurality of posts are withdrawn from the plurality of receptacles to permit rotation of the coupler cover relative to the camera housing. This example implementation is shown in FIG. 5.

FIG. 5 illustrates a partial perspective exploded view of an endoscopic camera assembly 500, in accordance with an example implementation. The endoscopic camera assembly 500 includes a camera housing 502 that is similar to the camera housing 112. The camera housing 502 has a conical section 504 at its distal end, and the conical section 504 differs from the conical portion 202 in that the conical section 504 has a plurality of receptacles (e.g., holes) such as receptacle 506 formed in a distal end face 508 of the conical section 504.

The endoscopic camera assembly 500 further includes a coupler cover 510 having a window 509 and a window 511. The coupler cover 510 also has a plurality of posts, such as post 512, disposed on its proximal end face.

The coupler cover 510 is configured to move between a proximal position in which the posts (e.g., the post 512) are received in the receptacles (e.g., the receptacle 506) of the camera housing 502. In this position, the coupler cover 510 is locked into the camera housing 502 and is precluded from changing its rotary position.

In an example, the endoscopic camera assembly 500 can have one or more springs such as spring 514 and spring 516, that bias the coupler cover 510 toward the proximal position to maintain the coupler cover 510 at the proximal, locked position. For example, the springs 514, 516 can be interposed between the coupler cover 510 and the coupling 110 described above with respect to FIGS. 1A, 1B.

If a user wants to change the rotary position of the coupler cover 510 to change the orientation of the windows 509, 511, the user can pull the coupler cover 510 (e.g., against the biasing force of the springs 514, 516) away from the camera housing 502, causing the posts of the coupler cover 510 to disengage from the respective receptacles of the camera housing 502. The user can then rotate the coupler cover 510 to a desired rotary/angular position, and re-engage the posts of the coupler cover 510 with the receptacles of the camera housing 502 (e.g., release the coupler cover 510 and allow the springs 514, 516 to push it in the proximal direction) to lock the coupler cover 510 at the new rotary/angular position.

In another example, the posts and receptacles may be reversed. In other words, the camera housing may have the posts, whereas the coupler cover may have the receptacles. In another example, rather than the camera housing, the coupling 110 may have the receptacles or the posts that engage with the coupler cover to lock the coupler cover in place.

The examples above describe changing an angular position of the coupler cover with respect to the camera housing. However, in other examples, an angular position of the coupler cover 206 is permanently fixed relative to the camera housing 112.

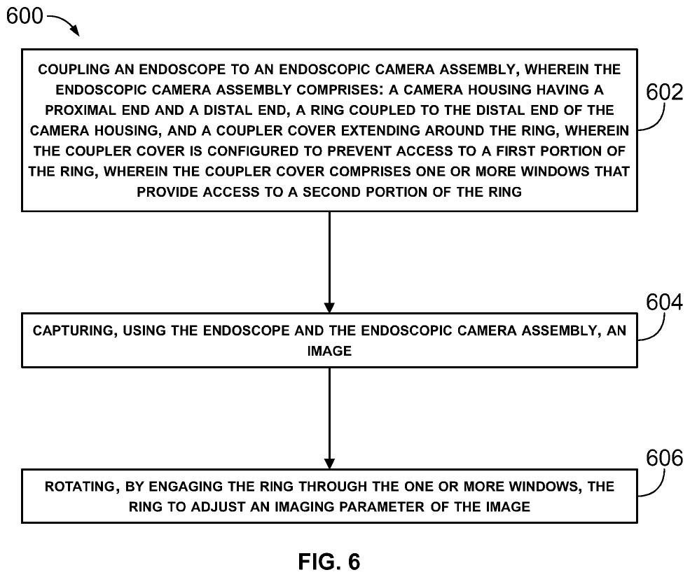

FIG. 6 is a flowchart of a method 600 for operating the endoscopic camera system 100, in accordance with an example implementation. The method 600 can, for example, be used with the endoscopic camera system 100 and the endoscopic camera assembly 108.

The method 600 may include one or more operations, or actions as illustrated by one or more of blocks 602-606, 700, 800, 900, 1000-1002, 1100, 1200, and 1300. Although the blocks are illustrated in a sequential order, these blocks may also be performed in parallel, and/or in a different order than those described herein. Also, the various blocks may be combined into fewer blocks, divided into additional blocks, and/or removed based upon the desired implementation. It should be understood that for this and other processes and methods disclosed herein, flowcharts show functionality and operation of one possible implementation of present examples. Alternative implementations are included within the scope of the examples of the present disclosure in which functions may be executed out of order from that shown or discussed, including substantially concurrent or in reverse order, depending on the functionality involved, as would be understood by those reasonably skilled in the art.

At block 602, the method 600 includes coupling the endoscope 102 to an endoscopic camera assembly (e.g., the endoscopic camera assembly 108, 500), wherein the endoscopic camera assembly 108 comprises: a camera housing (e.g., the camera housing 112, 502) having a proximal end and a distal end. the ring 204 coupled to the distal end of the camera housing, and a coupler cover (e.g., the coupler cover 206. 510) extending around the ring 204, wherein the coupler cover is configured to prevent access to a first portion of the ring 204, wherein the coupler cover comprises one or more windows (e.g., the windows 410, 412 or the windows 509, 511) that provide access to a second portion of the ring 204.

At block 604, the method 600 includes capturing, using the endoscope 102 and the endoscopic camera assembly, an image (e.g., an image in a cavity inside a patient).

At block 606, the method 600 includes rotating, by engaging the ring 204 through the one or more windows, the ring 204 to adjust an imaging parameter of the image.

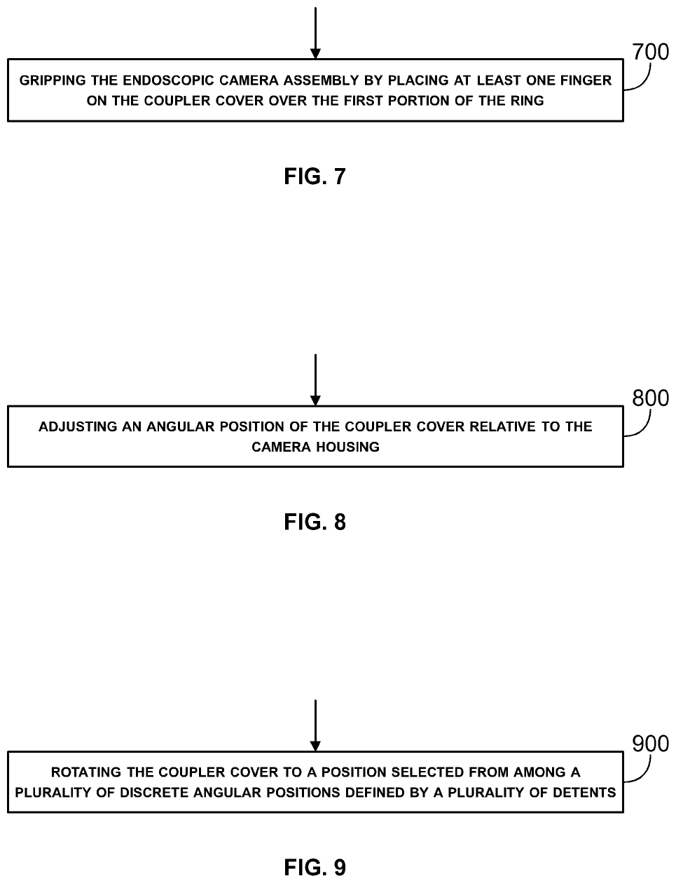

FIG. 7 is a flowchart of additional operations that are executable with the method 600, in accordance with an example implementation. At block 700, the operations include gripping the endoscopic camera assembly by placing at least one finger on the coupler cover over the first portion of the ring 204. The finger of the user does not unintentionally change the imaging parameter, as it does not have access to the first portion.

FIG. 8 is a flowchart of additional operations that are executable with the method 600, in accordance with an example implementation. At block 800, the operations include adjusting an angular position of the coupler cover relative to the camera housing. Different mechanisms are described above related to changing the rotary or angular position of the coupler cover 206, 510 when desired.

FIG. 9 is a flowchart of additional operations that are executable with the method 600, in accordance with an example implementation. In an example, adjusting the angular position of the coupler cover relative to the camera housing includes, at block 900, rotating the coupler cover to a position selected from among a plurality of discrete angular positions defined by a plurality of detents.

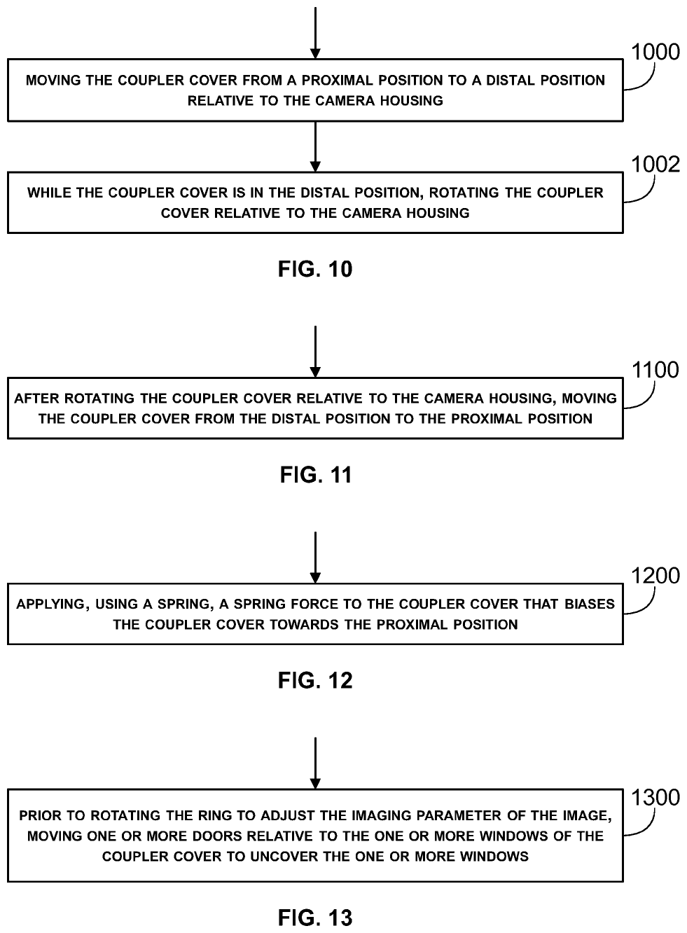

FIG. 10 is a flowchart of additional operations that are executable with the method 600, in accordance with an example implementation. In an example, adjusting the angular position of the coupler cover relative to the camera housing includes, at block 1000, moving the coupler cover from a proximal position relative to a distal position relative to the camera housing to disengage the coupler cover from the camera housing. At block 1002, the operations include, while the coupler cover is in the distal position, rotating the coupler cover relative to the camera housing.

In an example, as described above with respect to FIG. 5, one of the coupler cover and the camera housing comprises a plurality of posts, the other one of the coupler cover and the camera housing comprises a plurality of receptacles, and the plurality of receptacles are configured to receive the plurality of posts. In the proximal position, the plurality of posts are received in the plurality of receptacles to prevent rotation of the coupler cover relative to the camera housing. In the distal position, the plurality of posts are withdrawn from the plurality of receptacles to permit rotation of the coupler cover relative to the camera housing.

FIG. 11 is a flowchart of additional operations that are executable with the method 600, in accordance with an example implementation. At block 1100, the operations include, after rotating the coupler cover relative to the camera housing, moving the coupler cover from the distal position to the proximal position.

FIG. 12 is a flowchart of additional operations that are executable with the method 600, in accordance with an example implementation. At block 1200, moving the coupler cover from the distal position to the proximal position comprises applying, using a spring (e.g., the springs 514, 516), a spring force to the coupler cover that biases the coupler cover towards the proximal position.

FIG. 13 is a flowchart of additional operations that are executable with the method 600, in accordance with an example implementation. At block 1300, prior to rotating the ring 204 to adjust the imaging parameter of the image, moving one or more doors (e.g., door 414) relative to the one or more windows of the coupler cover to uncover the one or more windows.

The detailed description above describes various features and operations of the disclosed systems with reference to the accompanying figures. The illustrative implementations described herein are not meant to be limiting. Certain aspects of the disclosed systems can be arranged and combined in a wide variety of different configurations, all of which are contemplated herein.

Further, unless context suggests otherwise, the features illustrated in each of the figures may be used in combination with one another. Thus, the figures should be generally viewed as component aspects of one or more overall implementations, with the understanding that not all illustrated features are necessary for each implementation.

Additionally, any enumeration of elements, blocks, or steps in this specification or the claims is for purposes of clarity. Thus, such enumeration should not be interpreted to require or imply that these elements, blocks, or steps adhere to a particular arrangement or are carried out in a particular order.

Further, devices or systems may be used or configured to perform functions presented in the figures. In some instances, components of the devices and/or systems may be configured to perform the functions such that the components are actually configured and structured (with hardware and/or software) to enable such performance. In other examples, components of the devices and/or systems may be arranged to be adapted to, capable of, or suited for performing the functions, such as when operated in a specific manner.

By the term “substantially” or “about” it is meant that the recited characteristic, parameter, or value need not be achieved exactly, but that deviations or variations, including for example, tolerances, measurement error, measurement accuracy limitations and other factors known to skill in the art, may occur in amounts that do not preclude the effect the characteristic was intended to provide.

The arrangements described herein are for purposes of example only. As such, those skilled in the art will appreciate that other arrangements and other elements (e.g., machines, interfaces, operations, orders, and groupings of operations, etc.) can be used instead, and some elements may be omitted altogether according to the desired results. Further, many of the elements that are described are functional entities that may be implemented as discrete or distributed components or in conjunction with other components, in any suitable combination and location.

While various aspects and implementations have been disclosed herein, other aspects and implementations will be apparent to those skilled in the art. The various aspects and implementations disclosed herein are for purposes of illustration and are not intended to be limiting, with the true scope being indicated by the following claims, along with the full scope of equivalents to which such claims are entitled. Also, the terminology used herein is for the purpose of describing particular implementations only, and is not intended to be limiting.

Embodiments of the present disclosure can thus relate to one of the enumerated example embodiment (EEEs) listed below.

EEE 1 is an endoscopic camera assembly, comprising: a camera housing having a proximal end and a distal end; a ring coupled to the distal end of the camera housing; and a coupler cover extending around the ring, wherein the coupler cover is configured to prevent access to a first portion of the ring, wherein the coupler cover comprises one or more windows that provide access to a second portion of the ring, and wherein the ring is rotatable relative to the camera housing and the coupler cover, such that rotation of the ring adjusts an imaging parameter of the endoscopic camera assembly.

EEE 2 is the endoscopic camera assembly of EEE 1, wherein the one or more windows comprise a first window and a second window, and wherein a center of the first window and a center of the second window are diametrically-opposite from each other on a circumference of the coupler cover.

EEE 3 is the endoscopic camera assembly of any of EEEs 1-2, wherein an angular position of the coupler cover relative to the camera housing is adjustable.

EEE 4 is the endoscopic camera assembly of EEE 3, wherein the coupler cover is coupled to the distal end of the camera housing by a plurality of detents that define a plurality of discrete angular positions of the coupler cover relative to the camera housing.

EEE 5 is the endoscopic camera assembly of EEE 3, wherein (i) one of the coupler cover and the camera housing comprises a plurality of posts, (ii) the other one of the coupler cover and the camera housing comprises a plurality of receptacles, and (iii) the plurality of receptacles are configured to receive the plurality of posts.

EEE 6 is the endoscopic camera assembly of EEE 5, wherein the coupler cover is configured to move between a proximal position relative to the camera housing and a distal position relative to the camera housing, wherein, in the proximal position, the plurality of posts are received in the plurality of receptacles to prevent rotation of the coupler cover relative to the camera housing, and wherein, in the distal position, the plurality of posts are withdrawn from the plurality of receptacles to permit rotation of the coupler cover relative to the camera housing.

EEE 7 is the endoscopic camera assembly of EEE 6, further comprising: one or more springs that bias the coupler cover towards the proximal position.

EEE 8 is the endoscopic camera assembly of any of claims 1-2, wherein an angular position of the coupler cover is permanently fixed relative to the camera housing.

EEE 9 is the endoscopic camera assembly of any of EEEs 1-8, wherein an exterior surface of the coupler cover comprises a grip portion that is substantially planar.

EEE 10 is the endoscopic camera assembly of any of EEEs 1-9, wherein a window of the one or more windows span an angle between 10 degrees and 80 degrees about a circumference of the coupler cover.

EEE 11 is the endoscopic camera assembly of any of EEEs 1-10, wherein the coupler cover comprises one or more doors that are movable relative to the one or more windows of the coupler cover, wherein each door is moveable relative to a respective window of the one or more windows between (i) an open position in which the door does not cover the respective window and (ii) a closed position in which the door covers the respective window.

EEE 12 is the endoscopic camera assembly of any of EEEs 1-11, wherein the one or more windows have an axial length in a dimension extending between a proximal end face of the coupler cover and a distal end face of the coupler cover, wherein the one or more windows have an arc length in a dimension extending around a circumference of the coupler cover, and wherein the arc length is greater than the axial length.

EEE 13 is the endoscopic camera assembly of any of EEEs 1-12, wherein the coupler cover is funnel-shape such that a proximal portion of the coupler cover interfacing with the camera housing is divergent.

EEE 14 is the endoscopic camera assembly of EEE 13, wherein the camera housing comprises a conical portion at the distal end of the camera housing, such that the conical portion interfaces with the proximal portion of the coupler cover that is a divergent.

EEE 15 is the endoscopic camera assembly of EEE 14, wherein the conical portion comprises a plurality of longitudinal grooves formed in a circular array about an exterior surface of the conical portion of the camera housing.

EEE 16 is a method of operating an endoscopic camera system, comprising: coupling an endoscope to an endoscopic camera assembly, wherein the endoscopic camera assembly comprises: a camera housing having a proximal end and a distal end, a ring coupled to the distal end of the camera housing, and a coupler cover extending around the ring, wherein the coupler cover is configured to prevent access to a first portion of the ring, wherein the coupler cover comprises one or more windows that provide access to a second portion of the ring; capturing, using the endoscope and the endoscopic camera assembly, an image; and rotating, by engaging the ring through the one or more windows, the ring to adjust an imaging parameter of the image.

EEE 17 is the method of EEE 16, further comprising: gripping the endoscopic camera assembly by placing at least one finger on the coupler cover over the first portion of the ring.

EEE 18 is the method of any of EEEs 16-17, further comprising: adjusting an angular position of the coupler cover relative to the camera housing.

EEE 19 is the method of EEE 18, wherein adjusting the angular position of the coupler cover relative to the camera housing comprises: rotating the coupler cover to a position selected from among a plurality of discrete angular positions defined by a plurality of detents.

EEE 20 is the method of EEE 18, wherein adjusting the angular position of the coupler cover relative to the camera housing comprises: moving the coupler cover from a proximal position to a distal position relative to the camera housing; and while the coupler cover is in the distal position, rotating the coupler cover relative to the camera housing.

EEE 21 is the method of EEE 20, wherein (i) one of the coupler cover and the camera housing comprises a plurality of posts, (ii) the other one of the coupler cover and the camera housing comprises a plurality of receptacles, and (iii) the plurality of receptacles are configured to receive the plurality of posts, wherein, in the proximal position, the plurality of posts are received in the plurality of receptacles to prevent rotation of the coupler cover relative to the camera housing, and wherein, in the distal position, the plurality of posts are withdrawn from the plurality of receptacles to permit rotation of the coupler cover relative to the camera housing.

EEE 22 is the method of any of EEEs 20-21, further comprising: after rotating the coupler cover relative to the camera housing, moving the coupler cover from the distal position to the proximal position.

EEE 23 is the method of claim 22, wherein moving the coupler cover from the distal position to the proximal position comprises: applying, using one or more springs, a spring force to the coupler cover to bias the coupler cover towards the proximal position.

EEE 24 is the method of any of EEEs 16-23, further comprising: prior to rotating the ring to adjust the imaging parameter of the image, moving one or more doors relative to the one or more windows of the coupler cover to uncover the one or more windows.

What is claimed is:

1. An endoscopic camera assembly, comprising:
a camera housing having a proximal end and a distal end;
a ring coupled to the distal end of the camera housing; and
a coupler cover extending around the ring,
wherein the coupler cover is configured to prevent access to a first portion of the ring.
wherein the coupler cover comprises one or more windows that provide access to a second portion of the ring. and
wherein the ring is rotatable relative to the camera housing and the coupler cover. such that rotation of the ring adjusts an imaging parameter of the endoscopic camera assembly.

2. The endoscopic camera assembly of claim 1, wherein the one or more windows comprise a first window and a second window, and
wherein a center of the first window and a center of the second window are diametrically-opposite from each other on a circumference of the coupler cover.

3. The endoscopic camera assembly of claim 1, wherein an angular position of the coupler cover relative to the camera housing is adjustable.

4. The endoscopic camera assembly of claim 3, wherein the coupler cover is coupled to the distal end of the camera housing by a plurality of detents that define a plurality of discrete angular positions of the coupler cover relative to the camera housing.

5. The endoscopic camera assembly of claim 3. wherein (i) one of the coupler cover and the camera housing comprises a plurality of posts, (ii) the other one of the coupler cover and the camera housing comprises a plurality of receptacles, and (iii) the plurality of receptacles are configured to receive the plurality of posts.

6. The endoscopic camera assembly of claim 5, wherein the coupler cover is configured to move between a proximal position relative to the camera housing and a distal position relative to the camera housing,
wherein, in the proximal position, the plurality of posts are received in the plurality of receptacles to prevent rotation of the coupler cover relative to the camera housing, and
wherein, in the distal position, the plurality of posts are withdrawn from the plurality of receptacles to permit rotation of the coupler cover relative to the camera housing.

7. The endoscopic camera assembly of claim 6, further comprising:
one or more springs that bias the coupler cover towards the proximal position.

8. The endoscopic camera assembly of claim 1, wherein an angular position of the coupler cover is permanently fixed relative to the camera housing.

9. The endoscopic camera assembly of claim 1, wherein an exterior surface of the coupler cover comprises a grip portion that is substantially planar.

10. The endoscopic camera assembly of claim 1, wherein a window of the one or more windows span an angle between 10 degrees and 80 degrees about a circumference of the coupler cover.

11. The endoscopic camera assembly of claim 1, wherein the coupler cover comprises one or more doors that are movable relative to the one or more windows of the coupler cover,
wherein each door is moveable relative to a respective window of the one or more windows between (i) an open position in which the door does not cover the respective window and (ii) a closed position in which the door covers the respective window.

12. The endoscopic camera assembly of claim 1, wherein the one or more windows have an axial length in a dimension extending between a proximal end face of the coupler cover and a distal end face of the coupler cover,
wherein the one or more windows have an arc length in a dimension extending around a circumference of the coupler cover, and
wherein the arc length is greater than the axial length.

13. The endoscopic camera assembly of claim 1, wherein the coupler cover is funnel-shape such that a proximal portion of the coupler cover interfacing with the camera housing is divergent.

14. The endoscopic camera assembly of claim 13, wherein the camera housing comprises a conical portion at the distal end of the camera housing, such that the conical portion interfaces with the proximal portion of the coupler cover that is a divergent.

15. The endoscopic camera assembly of claim 14, wherein the conical portion comprises a plurality of longitudinal grooves formed in a circular array about an exterior surface of the conical portion of the camera housing.

16. A method of operating an endoscopic camera system, comprising:
coupling an endoscope to an endoscopic camera assembly, wherein the endoscopic camera assembly comprises:
a camera housing having a proximal end and a distal end,
a ring coupled to the distal end of the camera housing, and
a coupler cover extending around the ring, wherein the coupler cover is configured to prevent access to a first portion of the ring, wherein the coupler cover comprises one or more windows that provide access to a second portion of the ring;
capturing, using the endoscope and the endoscopic camera assembly, an image; and
rotating, by engaging the ring through the one or more windows, the ring to adjust an imaging parameter of the image.

17. The method of claim 16, further comprising:
gripping the endoscopic camera assembly by placing at least one finger on the coupler cover over the first portion of the ring.

18. The method of claim 16, further comprising:
adjusting an angular position of the coupler cover relative to the camera housing.

19. The method of claim 18, wherein adjusting the angular position of the coupler cover relative to the camera housing comprises:
rotating the coupler cover to a position selected from among a plurality of discrete angular positions defined by a plurality of detents.

20. The method of claim 18, wherein adjusting the angular position of the coupler cover relative to the camera housing comprises:
moving the coupler cover from a proximal position to a distal position relative to the camera housing; and
while the coupler cover is in the distal position, rotating the coupler cover relative to the camera housing.

21. The method of claim 20, wherein (i) one of the coupler cover and the camera housing comprises a plurality of posts, (ii) the other one of the coupler cover and the camera housing comprises a plurality of receptacles, and (iii) the plurality of receptacles are configured to receive the plurality of posts,
wherein, in the proximal position, the plurality of posts are received in the plurality of receptacles to prevent rotation of the coupler cover relative to the camera housing. and
wherein, in the distal position, the plurality of posts are withdrawn from the plurality of receptacles to permit rotation of the coupler cover relative to the camera housing.

22. The method of claim 20, further comprising:
after rotating the coupler cover relative to the camera housing, moving the coupler cover from the distal position to the proximal position.

23. The method of claim 22, wherein moving the coupler cover from the distal position to the proximal position comprises:
applying, using one or more springs, a spring force to the coupler cover to bias the coupler cover towards the proximal position.

24. The method of claim 16. further comprising:
prior to rotating the ring to adjust the imaging parameter of the image. moving one or more doors relative to the one or more windows of the coupler cover to uncover the one or more windows.
